(19) United States
(12) Patent Application Publication
(10) Pub. No.: US 2024/0164685 A1
(43) Pub. Date: May 23, 2024

(54) HEALTH MONITORING DEVICE

(71) Applicant: InfoBionic, Inc., Waltham, MA (US)

(72) Inventors: Lev KORZINOV, San Diego, CA (US); Eric BAUMANN, San Diego, CA (US); Anna MCNAMARA, Susquehanna, PA (US)

(73) Assignee: InfoBionic, Inc., Waltham, MA (US)

(21) Appl. No.: 18/425,683

(22) Filed: Jan. 29, 2024

Related U.S. Application Data
(63) Continuation of application No. 16/785,744, filed on Feb. 10, 2020, now Pat. No. 11,931,154.
Application No. 16/785,744 is a continuation of application No. 15/953,996, filed on Apr. 16, 2018, now Pat. No. 10,595,737.
Application No. 15/953,996 is a continuation of application No. 15/143,016, filed on Apr. 29, 2016, now Pat. No. 9,968,274.

(51) Int. Cl. A61B 5/316; A61B 5/00; A61B 5/25; A61B 5/364; H04B 1/3827; H04L 43/04; H04W 4/70

(52) U.S. Cl. CPC A61B 5/316; A61B 5/0006; A61B 5/0022; A61B 5/25; A61B 5/364; A61B 5/7246; A61B 5/7264; A61B 5/7475; H04B 1/385; H04L 43/04; H04W 4/70; A61B 5/02405; A61B 5/7203; A61B 5/7282

(57) ABSTRACT

A method for processing ECG data may include: receiving ECG data representing heartbeats; determining whether each of the heartbeats is normal or abnormal; associating each abnormal heartbeat with an existing or new template; receiving, from a user, input related to each new template that includes either: a) a confirmation that the new template represents an abnormal heartbeat, or b) a reclassification of the new template as representing a normal heartbeat or a different abnormal heartbeat; and in response to the user input, updating a label of each of the heartbeats associated with each confirmed new template and each of the heartbeats associated with each reclassified new template. The ECG data may be received from a portable monitor configured to be carried on a patient's body.

### TECHNICAL FIELD

Various embodiments of the present disclosure relate to a device and systems and methods of using the device for health monitoring, and more particularly to a device and system and methods of using a device for physiologic data monitoring.

### BACKGROUND

Physiologic data may be used to monitor the health of a patient. For example, bioelectric signals (e.g., electrocardiogram or ECG signals) from the patient's heart may be used to monitor cardiac health. ECG is a recording of the electrical activity of the heart. During ECG monitoring, electrodes attached to a patient's skin are used to detect electrical activity of the heart over a period of time, and electrical impulses generated by the heart during each heartbeat are detected and recorded and/or displayed on a device. Analysis of the data reveals the cardiac health (e.g., rate and regularity of heartbeats, size and position of the chambers, the presence of any damage to the heart, effects of drugs or devices used to regulate the heart, etc.) of the patient.

Multiple electrodes (e.g., left arm (LA), right arm (RA), and left leg (LL) electrodes) may be attached to the patient's skin for ECG measurement. These electrodes may be combined into a number of pairs (e.g., three pairs LA-RA, LA-LL, and RA-LL), and voltage signals may be recorded across each pair. Each pair is known as a lead. Each lead looks at the heart from a different angle. Different types of ECG measurements can be referred to by the number of leads that are recorded (e.g., 3-lead, 5-lead, 12-lead ECG, etc.).

Many cardiac problems become noticeable only during physical activity (walking, exercise, etc.). An ambulatory electrocardiogram (ECG) continuously monitors the electrical activity of the heart while a patient does normal activities. Typically, a 12-lead or a 5-lead ECG is used for periodic ECG monitoring (e.g., at a doctor's office, etc.) and a 3-lead ECG is used for continuous ambulatory monitoring. In 3-lead monitoring, ECG data is collected using three electrodes attached to the patient. The collected data is recorded in a monitor operatively coupled to the electrodes. The stored data is analyzed by a health care provider. In some cases, the monitor may transmit ECG data to a health care provider for analysis. Several types of monitors (e.g., Holter monitor, event monitors, mobile cardiovascular telemetry monitors, etc.) are known in the art. Some of these monitors store the data for subsequent analysis by a health care provider, while others transmit (real-time, periodically, or on demand) the collected ECG data to a remote site where it is analyzed.

### SUMMARY

Embodiments of the present disclosure relate to, among other things, devices for physiologic data monitoring. Each of the embodiments disclosed herein may include one or more of the features described in connection with any of the other disclosed embodiments.

A computer-implemented method for processing ECG data may include: receiving, over an electronic network, ECG data, wherein the ECG data represents a plurality of heartbeats; analyzing the ECG data, by at least one processor, to determine whether each of the plurality of heartbeats is a normal heartbeat or an abnormal heartbeat; associating, by the at least one processor, each of the abnormal heartbeats with either only one of a plurality of existing templates or a new template; receiving, from a user, input related to each new template, wherein the input includes either: a) a confirmation that the new template represents an abnormal heartbeat, or b) a reclassification of the new template as representing a normal heartbeat or a different abnormal heartbeat; and in response to the user input, updating, by the at least one processor, a label of each of the heartbeats associated with each confirmed new template and each of the heartbeats associated with each reclassified new template.

A system for processing ECG data may include a data storage device that stores instructions for processing ECG data; and a processor configured to execute the instructions to perform a method including: receiving, over an electronic network, ECG data, wherein the ECG data represents a plurality of heartbeats; analyzing the ECG data to determine whether each of the plurality of heartbeats is a normal heartbeat or an abnormal heartbeat; associating each of the abnormal heartbeats with either only one of a plurality of existing templates or a new template; receiving, from a user, input related to each new template, wherein the input includes either: a) a confirmation that the new template represents an abnormal heartbeat, or b) a reclassification of the new template as representing a normal heartbeat or a different abnormal heartbeat; and in response to the user input, updating the labels of each of the heartbeats associated with each confirmed new template and each of the heartbeats associated with each reclassified new template.

A non-transitory computer-readable medium may store instructions that, when executed by a computer, cause the computer to perform a method for processing ECG data, the method including: receiving, over an electronic network, ECG data, wherein the ECG data represents a plurality of heartbeats; analyzing the ECG data, by at least one processor, to determine whether each of the plurality of heartbeats is a normal heartbeat or an abnormal heartbeat; associating, by the at least one processor, each of the abnormal heartbeats with either only one of a plurality of existing templates or a new template; receiving, from a user, input related to each new template, wherein the input includes either: a) a confirmation that the new template represents an abnormal heartbeat, or b) a reclassification of the new template as representing a normal heartbeat or a different abnormal heartbeat; and in response to the user input, updating, by the at least one processor, a label of each of the heartbeats associated with each confirmed new template and each of the heartbeats associated with each reclassified new templates.

A method, system, or non-transitory computer-readable medium for processing ECG data may additionally or alternatively include one or more of the following steps or features: the method does not include repeating the analyzing step; the method may further comprise totaling, by the at least one processor, the number of heartbeats associated with the confirmed new templates and the number of heartbeats associated with the new templates reclassified as a different abnormal heartbeat; the associating step may include comparing ECG data representing a heartbeat to at least one of the plurality of existing templates; the step of associating may include creating the new template for abnormal heartbeats having characteristics that differ by more than a predefined threshold from each of the existing templates; the step of associating an abnormal heartbeat with one of the plurality of existing templates may include associating the abnormal heartbeat with an existing template if the abnormal heartbeat has characteristics that differ by less than a predefined threshold from the existing template; the abnormal heartbeats may include premature ventricular contractions; the electronic network may include a wireless connection over a cellular network; and the receiving step may include receiving the ECG data from a monitor, and the monitor may be a portable device configured to be carried on a patient's body.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates an exemplary system for measuring ECG of a patient.

FIG. 2 illustrates an exemplary device used in the ECG measurement system of FIG. 1.

FIGS. 3 and 4 illustrate steps in an exemplary process for detecting and classifying arrhythmias.

### DETAILED DESCRIPTION

### Overview of a System for Monitoring Physiologic Data

Embodiments of the present disclosure may include methods and systems for monitoring physiologic data of a patient. Various aspects of the present disclosure may be used in combination with, or include, one or more features disclosed in U.S. Pat. Nos. 8,478,418 (issued Jul. 2, 2013) and 8,620,418 (issued Dec. 31, 2013), each of which is incorporated by reference herein in its entirety. While an exemplary embodiment of measuring ECG data is described below, it should be noted that the current disclosure may be applied to the measurement of any physiologic data. For example, the disclosed systems and methods may be used to measure signals indicative of heart rate, activity level (e.g., physical mobility or movement), respiration rate, blood pressure (e.g., systolic and/or diastolic), blood oxygen saturation (SpO2), blood glucose or insulin level, pulse oximetry, impedance, body temperature, etc. Thus, the systems, devices, and methods described herein may acquire and process other types of physiologic data instead of or in addition to ECG data. It is also contemplated that, in some embodiments, the measured physiologic data may be used to determine a cardiac safety indicator such as QT prolongation, ST elevation, etc.

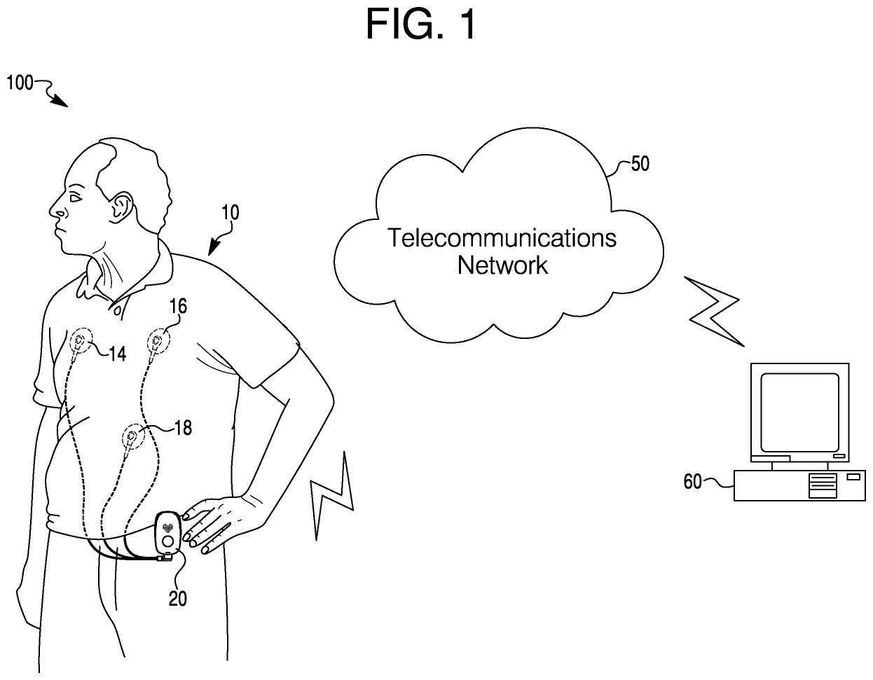

FIG. 1 is a schematic illustration of an exemplary system 100 for measuring ECG of a patient 10. A plurality of electrodes 14, 16, 18 may be attached to the patient 10 to detect ECG signals. Although a three-electrode configuration is illustrated, electrodes may be placed to measure any number of leads (e.g., a 10 electrode, 12-lead configuration). In one example, the electrodes 14, 16, 18 acquire two leads (channels) of ECG data. The electrodes 14, 16, 18 detect (and in some cases amplify) tiny electrical changes on the skin that are caused when heart muscles depolarize during each heartbeat. At rest, each heart muscle cell has a negative charge (called the membrane potential) across its cell membrane. Decreasing this negative charge toward zero, via the influx of the positive cations (Na+ and Ca++) is called depolarization. Depolarization activates mechanisms in the cell that cause it to contract. During each heartbeat, a healthy heart will have an orderly progression of a wave of depolarization that is triggered by the cells in the sinoatrial node, spreads out through the atrium, passes through the atrioventricular node and then spreads all over the ventricles. The depolarization wave (or ECG data) is indicative of the overall rhythm of the heart and is detected as variations in voltage between the electrode pairs (e.g., between electrodes 14-16, 14-18, and 16-18).

System 100 may include a monitor 20 operatively coupled to the electrodes 14, 16, 18. Monitor 20 may be adapted to receive and store the ECG data from the electrodes 14, 16, 18 using standard connections known in the art (e.g., lead wires, an analog to digital converter, etc.). In one example, the lead wires connected to each electrode in FIG. 1 may include a resistor. If a patient is undergoing defibrillation, the resistor may prevent the monitor from diverting energy applied to the patient by the defibrillation device. The presence of resistors in the lead wires does not inhibit impedance tomography. In one example, the resistor in each lead wire may be 1000 ohms.

In addition to the connection to electrodes 14, 16, 18, the monitor 20 may be configured to communicate with one or more additional or alternative sensors via wired or wireless connections. Any combination of well-known physiological sensors may be coupled to the monitor 20, such as SpO2 sensors, blood pressure sensors, heart electrodes (e.g., electrodes 14, 16, 18), respiration sensors, movement and activity sensors, glucose monitors, and the like. Respiration data may be derived from ECG baseline data, as is known to those of skill in the art. In one example, the monitor 20 can connect to a sensor in a scale to receive information related to the patient's weight. Movement or activity may be sensed with appropriate accelerometers or gyroscopes, which may include micro electro-mechanical system (MEMS) devices. The one or more additional or alternative sensors may be connected to the monitor 20 via wires or optical cables or via a wireless link (e.g., Bluetooth, Wi-Fi, ZigBee, Z-wave, radio, etc.).

In one example, at least one type of sensor transmits data to the monitor 20 via a wired connection, and at least one type of sensor transmits data to the monitor 20 via a wireless connection. The patient 10 may press a button on the monitor 20 to wirelessly pair it with one or more of the sensors described above. In another example, a user may communicate with a monitor 20 via a web/mobile interface component to wirelessly pair the monitor 20 with selected sensors.

In some embodiments, monitor 20 may transfer at least a portion of the measured ECG data (or other physiologic data) to a remote analysis station 60 for analysis. Although analysis station 60 is illustrated as a computer (e.g., processor and memory), in general, analysis station 60 may include any collection of computational devices (e.g., one or more servers, databases, and computers networked together) and personnel. The term “processor” as used herein may include a central processing unit or a microprocessor. The ECG data from monitor 20 may be transferred to remote analysis station 60 over a wired connection, using a portable storage medium (transferrable memory device, etc.), or wirelessly over a telecommunications network 50 (e.g., a cellular network, the Internet, a computer network, etc.). For example, monitor 20 may include a cellular modem, and the ECG data may be sent to the analysis station 60 via a cellular network. As used herein, the term “electronic network” may include any combination of wired and wireless communication technologies used to transmit information.

Analysis station 60 may analyze the ECG data to check the cardiac health of patient 10. Any analysis methodology known in the art may be used to analyze the received data (e.g., a methodology described by Philip de Chazal, et al., in “Automatic Classification of Heartbeats Using ECG Morphology and Heartbeat Interval Features,” IEEE Transactions on Biomedical Engineering, Vol. 51, No. 7, July, 2004). In some embodiments, monitor 20 may at least partially analyze the collected ECG data before it is transferred to analysis station 60.

In some embodiments, monitor 20 may store the collected ECG data, and continuously transmit (directly or through an intermediate device) a subset of the data (e.g., data at a lower resolution, etc.) to the analysis station 60. In one example, the subset of the data is transmitted at 100 samples per second, although it may be transmitted at 200 samples per second or at any other frequency. The analysis station 60 may analyze the received data to determine if it indicates an anomaly (e.g., an arrhythmia, an unexpected trend in the data, etc.). If an anomaly is indicated, analysis station 60 may request (i.e. transmit instructions) the monitor 20 for more data (e.g., data from the same time frame at a higher resolution, etc.). For example, if the initial data was transmitted at 100 samples per second, the second set of more detailed data may be transmitted at 200 samples per second. Upon receipt of this request, the monitor 20 may retrieve the requested data from memory and transmit it to the analysis station 60. The analysis station 60 may then analyze the data (e.g., using a more rigorous analysis methodology) to confirm or refute the anomaly detected during the previous analysis. This analysis methodology is described in more detail in U.S. Pat. No. 8,478,418, which is incorporated by reference herein.

### Monitor

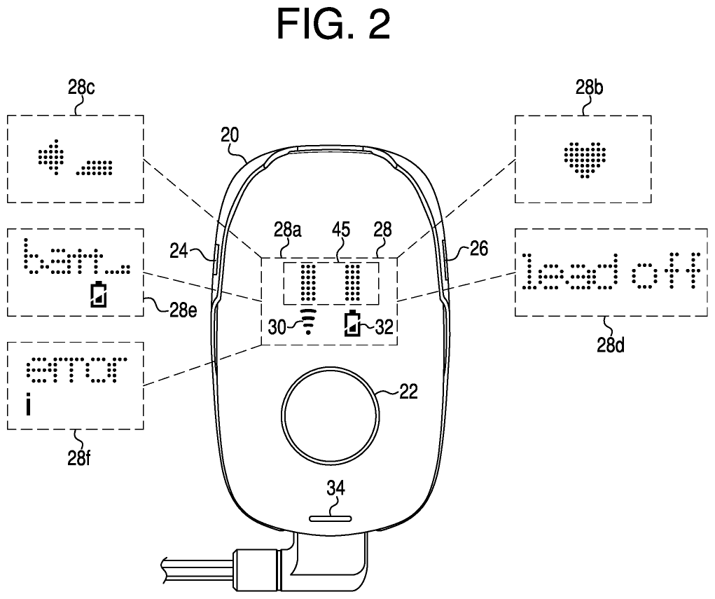

FIG. 2 illustrates an exemplary embodiment of monitor 20. Monitor 20 may include integrated circuits (microprocessor, memory, communication devices, etc.), visual displays (LED, LCD, etc.), and/or buttons that can be activated by the patient 10. The integrated circuits of monitor 20 may enable processing of collected ECG data, and communication between monitor 20 and the analysis station 60. The buttons may enable the patient 10 to trigger an activity (data collection, communication with analysis station 60, record or mark an event, etc.), and the display may enable the monitor 20 and analysis station 60 to communicate with patient 10 (e.g., using text messages). In one embodiment, the monitor 20 may include dimensions of approximately 108 mm×67 mm×17 mm, although the monitor 20 may be any size that allows it to be portable with the patient.

Monitor 20 may be a portable device, sized and adapted to be kept in the possession (strapped, attached, placed in the pocket, etc.) of patient 10. Such a portable monitor 20 may enable the patient 10 to go about the patient's daily activities while the monitor 20 records (and/or transfers, analyzes, etc.) ECG data. In the exemplary embodiment illustrated in FIG. 1, monitor 20 is shown as a device attached by a connector (e.g., clipped) to the patient's belt. However, this is only exemplary, and other configurations are possible (e.g., the connector could allow the device to be worn around the patient's neck). In embodiments where electrodes 14, 16, 18 are connected by a wire to the monitor, monitor 20 may include a connector to receive the connecting wire. In embodiments where electrodes 14, 16, 18 are coupled wirelessly, monitor 20 may include a transceiver to communicate with a transceiver of electrodes 14, 16, 18.

In one embodiment, the monitor 20 may include an event button 22, a wake button 24, and a volume button 26. A physician may press the event button 22 to activate the monitor 20 for patient use. Furthermore, the patient 10 may press the event button 22 if a symptom, such as the feeling caused by an arrhythmia, occurs. However, the monitor 20 may continuously record ECG data whether or not the patient presses the event button 22. Information from the event button 22 may therefore serve to help confirm suspected arrhythmias or other irregular heart activity detected from the ECG data. The wake button 24 may be pressed by the patient 10 to display the current level of reception (e.g., via a cellular network), the battery level, and/or whether the electrodes 14, 16, 18 are adequately coupled to the patient 10. Upon pressing the wake button 24, a light 34 may be green if the electrodes are all adequately coupled to the patient or red if one or more of the electrodes is not adequately coupled to the patient. The volume button 26 may allow the patient 10 to adjust or mute the volume of alerts from the monitor 20.

The monitor 20 may include a display 28. The display 28 may include a plurality of LED lights and one or more icons underneath the outer casing of the monitor. The LED lights may form an LED matrix 45 (e.g., 24×7, 20×7, or any other suitable arrangement of lights). In one embodiment, when the lights are off, the display is either imperceptible or faintly visible. When one or more LED lights or icons are lit, however, the individual lights or icons may be visible through the portion of the outer casing of the monitor that overlays the display 28. That portion of monitor 20 (a window over display 28) may be made of a transparent or semi-transparent material, for example, translucent polycarbonate.

A variety of display patterns may appear on the display 28 at various times to provide information to the patient 10. In FIG. 2, for example, the display pattern 28a may include a wireless icon 30 and a battery icon 32, which each correspond to one or more columns (e.g., three) of LED lights. Display pattern 28a may appear when the user (e.g., a physician, nurse, technician, patient, or any other person) presses the wake button 24. The columns of LED lights may indicate the level of wireless service and the battery level, respectively. Display pattern 28b may include an icon shaped like a heart, which may be displayed when the event button 22 is pressed. Display pattern 28c may include a speaker and rows of LED lights, and may be displayed when the user changes the volume. The number of rows of LED lights may increase when the volume is raised and decrease when the volume is lowered. “Mute” may be spelled in LED lights next to a speaker icon when the sound is muted. When one or more electrodes is not connected to the patient 10, the words “lead off” may scroll across the LED display, as shown in display pattern 28d. Alternatively, the words “lead” and “off” may alternate on the display 28. Display pattern 28e may appear when the battery is low. The number of rows of LED lights that appear may correspond to the level of battery remaining. Furthermore, the battery icon may appear red to indicate low battery status. In a final example, display pattern 28f may appear if there is an error that requires user attention. The LED lights may be lit in any suitable pattern or may form any words to communicate to the patient 10.

In other examples, display 28 may be separate from the monitor 20. The separate display 28 could be a stand-alone display or could be a user's cell phone or other communication device. The monitor 20 may transmit information to the stand-alone display, cell phone, or other communication device via any type of wireless network.

The monitor 20 may include a rechargeable battery. In one example, the battery may operate for between 24 and 72 hours on a single charge. The battery may be removable from the monitor 20 and docked to an external charger.

The hardware of monitor 20 may include various components connected by general purpose input/outputs or by specialized connectors. The hardware may include any suitable microprocessor and other circuitry known to one of ordinary skill in the art for performing the various functions of the system described herein, such as analog-to-digital converters, device/component drivers, tranceivers, and memory. The system software may receive ECG data for evaluation by an arrhythmia analysis algorithm, and any detected arrhythmias may be identified and presented for physician review. The system software may detect, for example, premature ventricular contractions (PVCs) from the ECG data, as will be described further below.

### Method for Processing of ECG Data

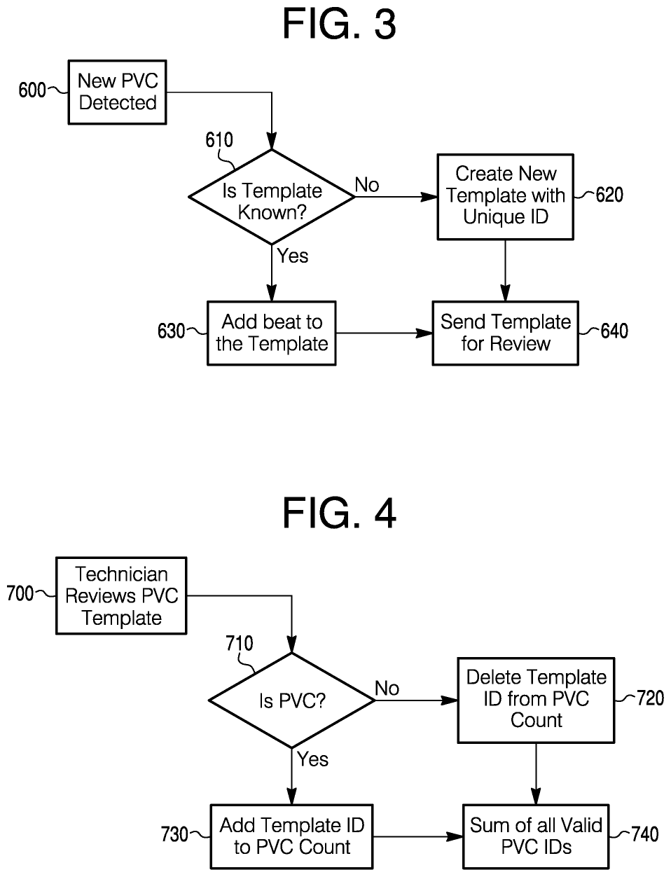

FIGS. 3 and 4 illustrate an automated method for processing ECG data to detect arrhythmias, or abnormal heartbeats. Although an exemplary embodiment of detecting premature ventricular contractions (PVCs) is described, the method can be used to detect any type of irregular heartbeat. The illustrated method may require less computational resources and may process ECG datasets with greater speed than existing methods of processing ECG data to detect PVCs. PVC detection may play a role in diagnosing a variety of heart conditions, including: heart attack, high blood pressure, cardiomyopathy (including congestive heart failure), disease of heart valves (such as mitral valve prolapse), hypokalemia (low blood levels of potassium), hypomagnesemia (low blood levels of magnesium), and hypoxia (low amounts of oxygen in the blood). In addition, several PVCs in a row with a high heart rate could indicate a serious arrhythmia, such as ventricular tachycardia.

Automatic classification of PVCs presents a challenge. For example, a patient may have over 100,000 heartbeats every day. Even if an automatic process/algorithm is 99% accurate, about 1000 beats per day may be misclassified. Time-consuming human validation may then be required to correct misclassifications. Some existing classification processes may require an entire ECG data set to be processed twice—a first processing to detect suspected PVCs and a second processing after receiving user input related to the suspected PVCs.

A method for detecting and classifying PVCs may include receiving ECG input/data. In one example, the original ECG data may be sampled at 1024 samples per second, although any other sampling rate may be used (e.g., 2048, 512, 256, etc.). The ECG input may undergo appropriate filtering and processing steps to, for example, eliminate noise; reduce the data to a lower number of samples per second, such as 200 or 100; and/or detect the amplitudes and/or thresholds of beats. This information may be used to detect PVCs. For example, PVC beats may have a higher amplitude than normal beats, and beats may be classified as PVC if they have a threshold over a fixed reference threshold.

Detected heartbeats may be labeled or classified as either: a) normal, or b) PVC. The PVC beats may be provided with a PVC template ID corresponding to their morphology (e.g., the duration and amplitude of the various waves/intervals/complexes). A PVC “template” is, for example, a representation of a suspected PVC beat that is derived from (e.g., is an average of) the characteristics of a plurality of suspected PVC beats having a similar morphology. Each template may therefore be associated with a plurality of beats having a certain morphology. Each template may have a unique template ID. Each patient 10 may have a plurality of different PVC templates, with each template corresponding to beats having a certain morphology.

FIG. 3 illustrates an exemplary method for determining whether to create a new PVC template. The method begins at step 600 with a suspected PVC beat that was detected, as described above. In step 610, the morphology of the suspected PVC may be compared to stored, previously-existing PVC templates (if there are any) to determine whether a template for the suspected PVC is known. The previously-existing PVC templates may be templates that were developed from earlier-processed heartbeats from the same patient. Additionally or alternatively, the previously-existing PVC templates may be based on known morphologies of PVCs, based on, for example, a population of patients. If the difference between the suspected PVC and the existing templates is above a certain threshold, a new PVC template may be created for the suspected PVC (step 620). In one example, the threshold may be 5% in absolute differences between one or more of the characteristics that define the morphology of a heartbeat (e.g., the duration and amplitude of the various waves/intervals/complexes). However, if the morphology of the suspected PVC is similar to an existing template (e.g., below a certain threshold), the PVC beat may be added to the existing template (step 630). In step 640, the templates are sent to a user for review.

FIG. 4 illustrates a method for user review of PVC templates. In step 700, the user reviews a PVC template. The user then determines whether the template is a PVC (step 710) and provides input to the hardware/software that analyzes the ECG data to determine arrhythmias. In one example, the user may complete step 710 by reviewing the information from the template, such as the duration and amplitude of various waves/intervals/complexes (e.g., the QRS complex), and determining whether the heartbeat is irregular when compared to the patient's normal heartbeat. Additionally or alternatively, the user might compare the information from the template to other known information about irregular heartbeats. If the user determines that the template does not represent a PVC, the template ID may be deleted from the PVC count (step 720), along with all beats associated with that template ID. Furthermore, the labels associated with each heartbeat corresponding to the deleted PVC template may be updated to indicate that the heartbeats are not PVCs. However, if the user determines that the template does represent a PVC, the template ID may be added to the PVC count (step 730). In other words, if the template ID is confirmed as a PVC by the user, all beats associated with the template ID may be confirmed and added to the PVC count, and the labels associated with each confirmed heartbeat may be updated to indicate that the heartbeats are PVCs. The beats associated with all of the valid PVC template IDs may then be added to determine the total PVC count.

Accordingly, the ECG data may be processed once to detect potential PVCs. The remaining steps of the PVC processing method may then rely on the beat labels (e.g., normal or PVC with a template ID), which may be about 200 times smaller in data size compared to the original ECG data. Because the process of FIG. 4 relies on beat labels and eliminates the need for all of the ECG data to be reprocessed to classify and total PVCs based on the user's input, the process can be carried out more efficiently than previously existing classification methods.

At the end of a pre-defined interval (e.g., a day), the total number of PVC beats may be calculated. In one example, if the total number is more than a predefined threshold (e.g., 100), the PVC statistics may be displayed by one or more of the monitor 20 or by a device used by the clinician for review.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

1-23. (canceled)

24. A computer-implemented method for classifying ECG data, comprising:
determining that an individual heartbeat in ECG data is likely an irregular heartbeat;
generating a heartbeat template based on the individual heartbeat;
receiving user input, wherein the user input includes a confirmation that the heartbeat template is representative of an irregular heartbeat;
determining that at least one additional individual heartbeat in the ECG data matches the heartbeat template.

25. The computer-implemented method of claim 24, wherein the irregular heartbeat is a premature ventricular contraction or an arrhythmia.

26. The computer-implemented method of claim 24, wherein determining that the individual heartbeat is likely an irregular heartbeat is based on a comparison of an amplitude of the individual heartbeat to a reference threshold.

27. The computer-implemented method of claim 24, wherein the heartbeat template is generated in response to determining that the individual heartbeat has a difference from a pre-existing heartbeat template in a template database that is above a predetermined difference threshold.

28. The computer-implemented method of claim 24, further comprising:
storing the heartbeat template in a template database; and
selectively labelling each individual heartbeat in the ECG data as likely to be a normal heartbeat or an irregular heartbeat based on a comparison of each individual heartbeat with the template database.

29. The computer-implemented method of claim 28, further comprising:
using the labels of the individual heartbeats in the ECG data to determine a total number of irregular heartbeats within a predetermined period of time.

30. The computer-implemented method of claim 24, further comprising:
receiving the ECG data from a portable device configured to be carried on a patient's body.

31. A system for classifying ECG data, comprising:
a data storage device that stores instructions for processing ECG data; and
one or more processors configured to execute the instructions to perform operations, including:
determining that an individual heartbeat in ECG data is likely an irregular heartbeat;
generating a heartbeat template based on the individual heartbeat;
receiving user input, wherein the user input includes a confirmation that the heartbeat template is representative of an irregular heartbeat;
determining that at least one additional individual heartbeat in the ECG data matches the heartbeat template.

32. The system of claim 31, wherein the irregular heartbeat is a premature ventricular contraction or an arrhythmia.

33. The system of claim 31, wherein determining that the individual heartbeat is likely an irregular heartbeat is based on a comparison of an amplitude of the individual heartbeat to a reference threshold.

34. The system of claim 31, wherein the heartbeat template is generated in response to determining that the individual heartbeat has a difference from a pre-existing heartbeat template in a template database that is above a predetermined difference threshold.

35. The system of claim 31, wherein the operations further include:
storing the heartbeat template in a template database; and
selectively labelling each individual heartbeat in the ECG data as likely to be a normal heartbeat or an irregular heartbeat based on a comparison of each individual heartbeat with the template database.

36. The system of claim 35, wherein the operations further include:
using the labels of the individual heartbeats in the ECG data to determine a total number of irregular heartbeats within a predetermined period of time.

37. The system of claim 31, wherein the operations further include:
receiving the ECG data from a portable device configured to be carried on a patient's body.

38. A non-transitory computer-readable medium storing instructions that, when executed by a computer, cause the computer to perform operations for evaluating ECG data, the operations including:
determining that an individual heartbeat in ECG data is likely an irregular heartbeat;
generating a heartbeat template based on the individual heartbeat;
receiving user input, wherein the user input includes a confirmation that the heartbeat template is representative of an irregular heartbeat;
determining that at least one additional individual heartbeat in the ECG data matches the heartbeat template.

39. The non-transitory computer-readable medium of claim 38, wherein the irregular heartbeat is a premature ventricular contraction or an arrhythmia.

40. The non-transitory computer-readable medium of claim 38, wherein determining that the individual heartbeat is likely an irregular heartbeat is based on a comparison of an amplitude of the individual heartbeat to a reference threshold.

41. The non-transitory computer-readable medium of claim 38, wherein the heartbeat template is generated in response to determining that the individual heartbeat has a difference from a pre-existing heartbeat template in a template database that is above a predetermined difference threshold.

42. The non-transitory computer-readable medium of claim 38, wherein the operations further include:
storing the heartbeat template in a template database; and
selectively labelling each individual heartbeat in the ECG data as likely to be a normal heartbeat or an irregular heartbeat based on a comparison of each individual heartbeat with the template database.

43. The non-transitory computer-readable medium of claim 42, wherein the operations further include:
using the labels of the individual heartbeats in the ECG data to determine a total number of irregular heartbeats within a predetermined period of time.
